(19) United States
(12) Patent Application Publication
(10) Pub. No.: US 2003/0041863 A1
(43) Pub. Date: Mar. 6, 2003

(54) Dual balloon endotracheal tube assembly

(76) Inventor: Jackie Hargis, Keota, OK (US)

Correspondence Address: MANELLI DENISON & SELTER, 2000 M STREET NW SUITE 700, WASHINGTON, DC, 20036-3307, US

(21) Appl. No.: 10/201,196

(22) Filed: Jul. 24, 2002

Related U.S. Application Data
(60) Provisional application No. 60/315,035, filed on Aug. 28, 2001.

(51) Int. Cl. A61M 16/00

(52) U.S. Cl. 128/207.14

(57) ABSTRACT

An endotracheal tube assembly includes an endotracheal tube having an air passage therethrough. A first inflatable member is associated with the endotracheal tube and is constructed and arranged to be inflated to maintain a portion of the endotracheal tube in the trachea. A second inflatable member is associated with the endotracheal tube and is constructed and arranged to be inflated separate from the first inflatable member so as to maintain the portion of the endotracheal tube in the trachea in the event the first inflatable member fails.

This application is based on U.S. Provisional Patent Application No. 60/315,035, filed on Aug. 28, 2001, and claims the benefit thereof for priority purposes.

### FIELD OF THE INVENTION

The invention relates to endotracheal tubes and, more particularly, to an endotracheal tube assembly that eliminates the need for reintubation in the event a primary balloon of the endotracheal tube assembly ruptures or leaks during intubation.

### BACKGROUND OF THE INVENTION

An endotracheal tube is used when a person is not able to breathe without the assistance of a ventilator. The tube is inserted through the nose or mouth, down into the trachea and a ventilator is attached to the tube. This process is called intubation. After the patient is intubated, a conventional balloon at the base of the tube is inflated through a small port and via an air-filled syringe so as to anchor the tube place. The tube is then taped and secured around the head and neck further protection from accidentally removal (extubation).

At times, the balloon may rupture. In this case, if the person is not ready for extubation, the only alternative is to remove the tube and insert a new tube. This causes added trauma to the throat and trachea. The person is usually awake at this time, causing emotional trauma as well.

Another reason to extubate and re-intubate a patient is if the patient has a “leaky cuff” or inflation balloon. This can be very irritating to the patient, causing uncontrollable coughing which can lead to accidentally coughing up the tube. Again, if this occurs, the patient needs to be re-intubated.

Accordingly, there is a need to provide an improved endotracheal tube assembly that eliminates the need for re-intubation in the event a primary balloon of the endotracheal tube assembly ruptures or leaks while a patient is intubated.

### SUMMARY OF THE INVENTION

An object of the invention is to fulfill the need referred to above. In accordance with the principles of the present invention, this objective is achieved by providing an endotracheal tube assembly including an endotracheal tube having an air passage therethrough. A first inflatable member is associated with the endotracheal tube and is constructed and arranged to be inflated to maintain a portion of the endotracheal tube in the trachea. A second inflatable member is associated with the endotracheal tube and is constructed and arranged to be inflated separate from the first inflatable member so as to maintain the portion of the endotracheal tube in the trachea in the event the first inflatable member fails.

Other objects, features and characteristics of the present invention, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawings, wherein like reference numerals refer to like parts, in which:

FIG. 1 is front view of a first embodiment of an endotracheal tube assembly provided in accordance with the principles of the present invention.

FIG. 2 is front view of a second embodiment of an endotracheal tube assembly provided in accordance with the principles of the present invention.

### DETAILED DESCRIPTION OF THE EXEMPLARY EMBODIMENTS

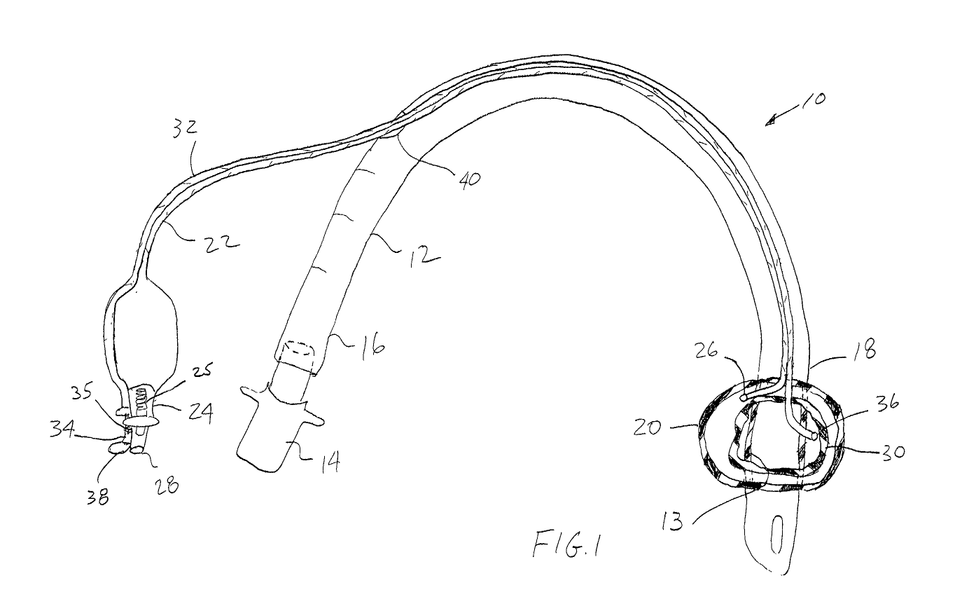

With reference to FIG. 1, a dual balloon endotracheal tube assembly, generally indicated at 10, is provided in accordance with the principles of the present invention. The assembly 10 includes the conventional flexible tube 12 having an airflow passage or lumen 13 therethrough. A respirator 14 is attached to a proximal end 16 of the tube 12. At the distal end 18 of the tube 12, a first or main inflatable member, such as a balloon 20, is provided about the tube 12. The first balloon 20 is inflated with air via inflation structure including a first inflation tube 22 connected to a first syringe port structure 24 for receiving a syringe (not shown) at end 28 to inflate the balloon 12. Thus, an end 26 of inflation tube 22 communicates with the interior of the first balloon 20 to inflate the first balloon 20 via a syringe. As shown, at least a portion of the inflation tube 22 is disposed within tube 12. The end 28 of the syringe port structure 24 has no stopper or cover thereon so that the user will know that this end ready to receive a syringe for use to inflate the first balloon 20 during a typical intubation procedure. The syringe port structure 24 includes a one-way valve 25 to prevent air from escaping to atmosphere.

A second, inflatable member, such as a balloon 30, is provided about the base of the tube 12, preferably within the interior of the first balloon 20. The second balloon 30 is inflated separately from the first balloon 20 via inflation structure including a second inflation tube 32 connected to a second syringe port structure 34. Thus, an end 36 of inflation tube 32 communicates with the interior of the second balloon 30 for inflation of the second balloon with air via a syringe (not shown) associated with the second syringe port structure 34. The second syringe port structure 34 also includes a one-way valve 35. As shown, at least a portion of the second inflation tube 32 is disposed within tube 12. The second balloon 30 is intended to be inflated only in the event the first balloon 20 fails. A pull-tab stopper 38 closes an open end of the syringe port structure 34 so that the user knows to use this syringe port structure 34 to inflate the second balloon 30. Thus the stopper 28 acts as an indicator, indicating to a user that this port is to inflate the second balloon 30. The stopper 28 and/or the syringe port structure 34 and/or the inflation tube 32 can be color-coded, (e.g., red) to indicate the association with the second balloon 30. The syringe port structure 24 and/or the inflation tube 22 can also be color-coded (e.g., blue) to indicate that these structures are used in the initial intubation procedure.

The second balloon 30 is shown to be inside the first balloon 20 but it can be appreciated that the first and second balloons may be totally separate from each other.

In the embodiment of FIG. 1, the inflation tubes 22 and 32 are shown to be separate tubes but can be joined for at least a portion thereof so as to provide a single structure entering an opening 40 in the tube 12. This makes insertion of the inflation tubes into the tube 12 easy.

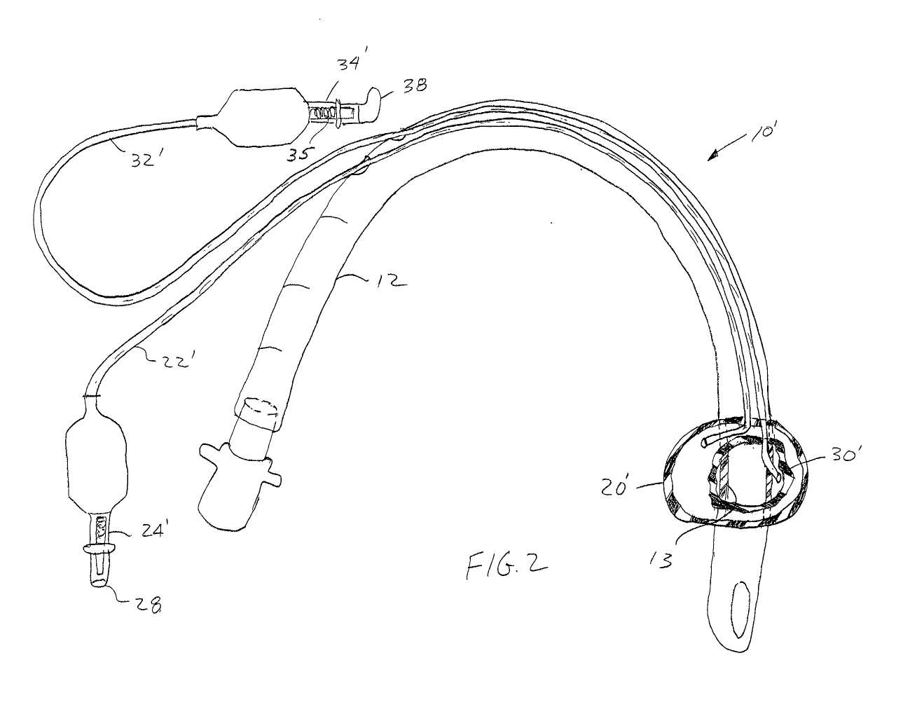

FIG. 2 shows a second embodiment of the invention, wherein the assembly 10′ includes completely separate systems for inflating the balloons. Thus, the first balloon 20′ is connected to an inflation tube 22′ and syringe port structure 24′ while the second balloon 30′ is connected to a separate inflation tube 32′ and syringe port structure 34′.

Thus, the second balloon 30 of the assembly 10 provides a means of keeping the end of an endotracheal tube 12 in place in a trachea even if the main balloon 20 fails. In the event the first balloon fails, the second balloon is simply inflated without removing the tube 12. Advantageously, re-intubation is not required, reducing trauma to the patient.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. An endotracheal tube assembly comprising:
an endotracheal tube having an air passage therethrough,
a first inflatable member associated with the endotracheal tube constructed and arranged to be inflated to maintain a portion of the endotracheal tube in the trachea; and
a second inflatable member associated with the endotracheal tube and being constructed and arranged to be inflated separate from the first inflatable member so as to maintain the portion of the endotracheal tube in the trachea in the event the first inflatable member fails.

2. The assembly of claim 1, wherein the first and second inflatable members are balloons.

3. The assembly of claim 1, further comprising first and second inflation structure, the first inflation structure being constructed and arranged to permit inflation of the first inflatable member and the second inflation structure being constructed and arranged to permit inflation of the second inflatable member.

4. The assembly of claim 3, wherein each of the first and second inflation structures includes an inflation tube having one end in communication with an interior of an associated inflatable member and another end in communication with a syringe port structure.

5. The assembly of claim 4, wherein at least a portion of each inflation tube is disposed within the endotracheal tube, the inflation tubes being at least partially joined so as to define a single structure entering the endotracheal tube.

6. The assembly of claim 4, wherein at least a portion of each inflation tube is disposed within the endotracheal tube, and wherein the inflation tubes are completely separated from each other.

7. The assembly of claim 1, wherein the second inflatable member is disposed within an interior of the first inflatable member.

8. The assembly of claim 1, further comprising a first syringe port structure and a first inflation tube associated with the first inflatable member for inflation thereof, and a second syringe port structure and a second inflation tube associated with the second inflatable member for inflation thereof, an indicator being associated with the second syringe port structure to indicate that the second syringe port structure is associated with inflating the second inflatable member.

9. The assembly of claim 8, wherein the indicator is a cover on the second syringe port structure.

10. The assembly of claim 8, wherein each syringe port structure includes a one-way valve.

11. An endotracheal tube assembly comprising:
an endotracheal tube having an air passage therethrough,
first inflatable means, associated with the endotracheal tube, for maintaining a portion of the endotracheal tube in the trachea; and
second inflatable means, associated with the endotracheal tube and inflatable separate from the first inflatable means, for maintaining the portion of the endotracheal tube in the trachea in the event the first inflatable means fails.

12. The assembly of claim 11, wherein the first and second inflatable means are balloons.

13. The assembly of claim 11, further comprising first means for permitting inflation of the first inflatable means and second means for permitting inflation of the second inflatable means.

14. The assembly of claim 13, wherein each of the first and second inflation permitting means includes an inflation tube having one end in communication with an interior of an associated inflatable means and another end in communication with a syringe port structure.

15. The assembly of claim 14, wherein at least a portion of each inflation tube is disposed within the endotracheal tube, the inflation tubes being at least partially joined so as to define a single structure entering the endotracheal tube.

16. The assembly of claim 13, wherein at least a portion of each inflation tube is disposed within the endotracheal tube, and wherein the inflation tubes are completely separated from each other.

17. The assembly of claim 11, wherein the second inflatable means is disposed within an interior of the first inflatable means.

18. The assembly of claim 11, further comprising a first syringe port structure and a first inflation tube associated with the first inflatable member, and a second syringe port structure and a second inflation tube associated with the second inflatable member, the assembly further comprising indicator means, associated with the second syringe port structure for indicating that the second syringe port structure is associated with inflating the second inflatable member.

19. The assembly of claim 18, wherein the indicator means is a cover on the second syringe port structure.

20. The assembly of claim 18, wherein the each syringe port structure includes a one-way valve.
